(19) United States
(12) Patent Application Publication
(10) Pub. No.: US 2009/0023685 A1
(43) Pub. Date: Jan. 22, 2009

(54) Nebulized or Sprayed Cidofovir for Recurrent Respiratory Papillomatosis

(76) Inventors: B. Louise Giles, Winnipeg (CA); Blair Seifert, Winnipeg (CA)

Correspondence Address: ADE & COMPANY INC., 2157 Henderson Highway, WINNIPEG, MB, R2G1P9, CA

(21) Appl. No.: 12/168,082

(22) Filed: Jul. 4, 2008

Related U.S. Application Data
(60) Provisional application No. 60/948,877, filed on Jul. 10, 2007.

(51) Int. Cl. A61K 31/675; A61P 11/00

(52) U.S. Cl. 514/86

(57) ABSTRACT

A solution of 2.5-20% cidofovir is administered as droplets having a mean size of about 1 micron to about 20 microns either nebulized or as a spray to an individual having recurrent respiratory papillomatosis.

### PRIOR APPLICATION INFORMATION

The instant application claims the benefit of U.S. Provisional Patent Application 60/948,877, filed Jul. 10, 2007.

### BACKGROUND OF THE INVENTION

Recurrent Respiratory Papillomatosis (RRP) is a rare albeit severe airway management problem in the pediatric population. Extremely difficult to treat, patients usually undergo multiple surgical procedures and toxic systemic medications in an attempt to control their disease.

### SUMMARY OF THE INVENTION

According to a first aspect of the invention, there is provided a method of treating recurrent respiratory papillomatosis comprising orally administering a solution of 2.5-20% cidofovir as droplets having a mean size of about 1 micron to about 20 microns to an individual in need of such treatment.

### BRIEF DESCRIPTION OF THE DRAWINGS

FIG. 1: Pre-topical Cidofovir

FIG. 2: Topical & nebulized Cidofovir

FIG. 3: CT scans pre & post Cidofovir

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Recurrent Respiratory Papillomatosis (RRP) is a disease caused by a virus, the Human Papillomavirus (HPV). HPV, which causes warts, has many sub-types, some which are responsible for genital warts, abnormal changes on women's cervix leading to cervical cancer (therefore a sexually transmitted disease) and RRP. Traditionally, the treatment for these warts (papillomas) has been surgical removal.

Surgical removal of papillomas in the airways must occur in the operating room with the patient under general anesthetic. Typically, however, the papillomas recur. Most patients require only infrequent removal of papillomas, especially those whose papillomas only occur on their vocal cords, only undergoing surgical removal when symptoms of the papilloma affects voice quality or becomes an obstruction to breathing. A minority of these patients have papillomas below their vocal cords, which infect the lower airways and even the lung itself. For those patients, breathing is severely affected, operations to remove the papillomas more risky, and subsequently, there is a high chance of developing lung cancer leading to untimely death.

As will be apparent to one of skill in the art, surgical procedures in the airways involve considerable risk. First, there is the risk of the anesthetic itself. Second, when one uses surgical instruments in the airway, the airway itself is partially blocked, reducing airflow and oxygen to the lungs. The airways are vascular and can bleed easily. In addition, most surgical procedures to remove papillomas necessitate the use of rigid hollow tubes which realistically cannot reach into the lower airways (which branch from the main airway, the trachea). This means that papillomas in the lower airways are not easily reached by conventional methods, and if attempted carries higher risk to the patient. Furthermore, the smaller the patient, the greater the risk.

Adjunctive therapy has been tried for papillomas in the airways with limited success. The best success has occurred with injecting an antiviral drug, cidofovir, directly into the papillomas at the vocal cord level. However, direct injection into papillomas in the lower airways is close to impossible due to surgical constraints.

Topical cidofovir has been used as a gel to treat lesions on the skin. In AIDS patients, gel-based cidofovir has been used to control herpes skin infections with success. However, the gel cannot be used in the airways due to its chemical composition.

For our patient, her small size made surgical removal of the papillomas in her lower airways close to impossible, injecting cidofovir impossible and she was failing intravenous therapy.

We decided that applying cidofovir using a flexible bronchoscope and a cytology brush soaked in cidofovir would be a reasonable method to attempt to deliver the drug to the papillomas. We used the intravenous form of the drug, diluted to the concentration used in the gel based formula. Initially, the drug did not stick well to the brush, so we thickened it with glycerine so that the drug would “stick” first to the brush, then to the papilloma. This preparation worked to reduce the size of the papillomas.

Although a success, the topical application of cidofovir was not without risk. The papillomas required “brushing” every 2 weeks to continue to see the effect of the drug. This required a general anesthetic every 2 weeks, which as discussed above is not without risk to the patient. Additionally, the application of the cytology brush through a flexible bronchoscope (which when in use reduced the patient's effective airway size to <25%) could potentially lead to complications such as hypoxia (low oxygen levels) and bleeding. Additionally, at one point the cytology brush got ‘stuck’ in the papilloma and was difficult to remove. We clearly needed a better strategy.

Other airway diseases, such as asthma, are treated with inhaled drugs, such as steroids. Infections of the airways in patients with Cystic Fibrosis are treated with nebulized antibiotics. We thought, why not this? Our Clinical Pharmacologist contacted the maker of cidofovir, who refused to discuss options for delivering the drug in this fashion. Therefore, we did it ourselves.

Nebulized drugs are administered by breaking up a solution of drug in a medication cup that has forced air going through it, making a “mist”. If the amount of solution in the medication cup has 4 ml in it, the resulting droplet size is 2-4 microns in diameter, the right size to deposit the drug into the airways after the main airway (trachea). Some drug will deposit more proximal (trachea) but the bulk of the drug deposits in the lower airways. Therefore, we made a 4 ml solution at the same concentration we were using for the topical application. This had numerous benefits for our patient: no more frequent trips to the operating room, the drug could be delivered more frequently, the drug could be administered at home, and obvious reduction of significant operative risks. Additionally, the drug could reach those airways which were unreachable by surgical means.

The nebulized drug worked well. It reached those areas previously unreachable. In fact, we even saw improvement in the lung (as evidenced by CT scans of the lung). However, as predicted, based on droplet size, the papillomas in the main airway (trachea) remained large. In our first patients' case, this was tackled with surgical removal and the topical application of our glycerine based cidofovir to the bare base of the papillomas. She is now papilloma free.

The nebulized form of cidofovir is meant for application of the drug in those patients whose disease is in the lower airways. However, to administer this drug, in this fashion, to those who only have papillomas on their vocal cords (or higher in the airway) is inappropriate. First, the drug goes to the wrong area of the airways. Second, this drug is not without known side effects: for example, the main side effect we encountered was bleeding (dose dependent & related to infection with bacteria, typically Pseudomomas aeruginosa). We were also worried about potential bronchospasm (asthma like closure of the airways) as our patient did exhibit cough with the drug. To expose someone to significant side effects like this was unacceptable.

Consequently, we then developed a spray solution of cidofovir, using the same concentration as described above. In some embodiments, a flavouring agent, for example, an addition of mint flavouring (can be adjusted to meet individual tastes and as such other flavourings known in the art may be utilized in the invention). In a preferred embodiment, the dispenser includes a long nozzle, which can be used to target the spray directly toward the papillomas on the vocal cords (or higher) without the worry of a significant amount of drug reaching the trachea or lower airways. In these embodiments, the droplets of cidofovir may have an average size of about 16 microns±3 microns.

During flexible bronchoscopy, where an endoscope is placed via the nares (nose) or through the mouth, the Bronchoscopist must ‘freeze’ the vocal cords and the subsequent trachea and lower airways to diminish the cough reflex (think of a piece of food going the wrong way, one coughs violently to prevent the food from going into the lungs). As a Bronchoscopist, I squirt Xylocalne (the same drug the dentist uses to freeze the mouth), onto the vocal cords, then I spray Xylocalne immediately after passing the vocal cords to freeze the trachea and lower airways. Just freezing the vocal cords does not prevent the cough from the trachea.

Therefore, we know that the spray cidofovir will only affect the vocal cords, and the nebulized form mainly deposits in the lower airways. The spray form is for the patient with isolated papillomas at the level of the vocal cords or higher, the nebulized form is for those with distal airway & lung involvement.

The topical preparation is 10% based in sterile glycerine. Dosing is variable, but at the time of surgery.

The concentration for nebulization is 2.5%-20%. It is diluted in sterile normal (0.9%) saline (NaCl) in a Class II laminar flow biologic safely hood. Cytotoxic precautions are taken. Dosing is 2-3 times/week (Daily q Monday, Wednesday & Friday).

The concentration for spray is 2.5%-20%. It is diluted in sterile normal (0.9%) saline (NaCl) in a Class II laminar flow biologic safely hood. Cytotoxic precautions are taken. Flavouring is added to make it more palatable. Dosing is daily×5 (Monday-Friday).

In other embodiments, other pharmaceutically acceptable diluents compatible with cidofovir may be used in place of saline. It is noted that such agents will be readily apparent to one of skill in the art.

Accordingly, in one embodiment of the invention, there is provided a method of treating recurrent respiratory papillomatosis comprising orally administering a solution of 2.5-20% cidofovir as droplets having a mean size of about 1 micron to about 20 microns to an individual in need of such treatment.

As will be appreciated by one of skill in the art, as used herein, ‘an individual in need of such treatment’ is an individual who has or is suspected of having recurrent respiratory papillomatosis, that is, a human papillomavirus infection of the airway.

As discussed above, in a preferred embodiment, the droplets have a mean diameter of approximately 12-20 microns or of approximately 13-19 microns for treatment of papillomas proximal to the vocal cords.

In other embodiments, the droplets have a mean diameter of approximately 1-4 microns for treatment of the airways after or below the main airway (trachea).

It is of note that other suitable diameters for distribution to other parts of the airway may be used and are within the scope of the invention as these may be determined by routine experimentation.

As will be appreciated by one of skill in the art, 0.25-20% cidofovir represents an effective amount of cidofovir in that it will accomplish at least one of the following: reduction of papilloma size, longer periods of remission or being symptom-free, improved breathing as well as other benefits which will be readily apparent to one skilled in the art.

Herein we report the use of nebulized cidofovir to treat RRP disease in a young girl without significant side effects. E presented at 11 months of age with respiratory failure and stridor. She was found to have laryngeal papillomas; her lesions completely obscured her upper airway. A tracheostomy was done for airway protection and it was discovered that her disease extended distally. Multiple surgical procedures failed to alleviate the lesions. Usual medical therapy (Interferon and Cidofovir) did not halt the progression of the disease; in fact, her lesions worsened especially in the right main bronchus. We elected to apply topical Cidofovir to her lower airway lesions citing literature to suggest that this approach was useful in other viral based lesions. Initially, Cidofovir (modified in sterile glycerin) was applied directly via flexible bronchoscopy every 2 weeks with good results. The lesions regressed with this technique. However, the lung papillomas could not be treated by this method. Therefore, we elected to nebulize the drug (Cidofovir 10 mg/ml in NS, 4 cc, 3 times/week). Her only complication was hemoptysis that resolved with decreasing the dose. Her lower airway lesions responded currently her lower airway lesions have visually disappeared. She is maintained on Cidofovir 2 times/week without complications. Nebulized Cidofovir appears to be a safe practical treatment for severe RRP.

The invention will now be described by way of examples; however, the invention is not necessarily limited by the examples.

### EXAMPLE 1

### Nebulized Form

### Case Report:

Background: Miss E is an ex-prem (24 week GA) born to a 17 year old mom who had antenatal bleeding and had been given steroids prior to delivery. Her postnatal course was complicated by Respiratory distress syndrome (mechanical ventilation for 52 days, CPAP for 28 days and O₂ for 30 days), a patent ductus arteriousus (indomethacin & surgical ligation), hemodynamic instability necessitating inotropic support and renal failure. Following discharge home Miss E had severe GERD, (L) inguinal hernia repair and was described to have colic. She had a “poor” or “weak” cry and “mild” stridor that was presumed to be secondary to vocal cord paresis or paralysis (although not confirmed by direct laryngoscopy).

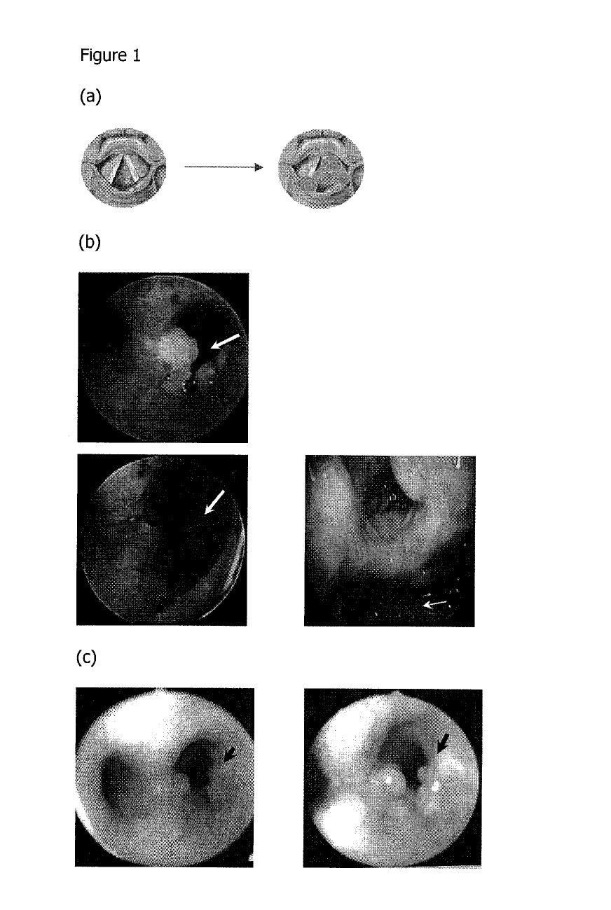

Presentation: At 10½ months of age, she presented to the Children's Hospital Emergency Room with increasing difficulties breathing. Her stridor had worsened, she had obvious shortness of breath and had an audible wheeze. She was admitted to hospital and subsequently transferred to the Pediatric intensive care unit. She was diagnosed with upper airway obstruction and treated with Heliox (80:20) and nebulized racemic epinephrine. She developed Type II respiratory failure (pCO2 62 mm Hg) and was intubated for further respiratory support. It was noted that she had “verrucous growths on vocal cords & aryepiglottic region”. Direct laryngoscopy & bronchoscopy confirmed the diagnosis of Papillomatosis and with lesions in the proximal trachea (FIG. 1a). She underwent tracheostomy. Pathology confirmed the diagnosis of papillomatosis due to Human Papillomavirus subtype 6 (HPV-6).

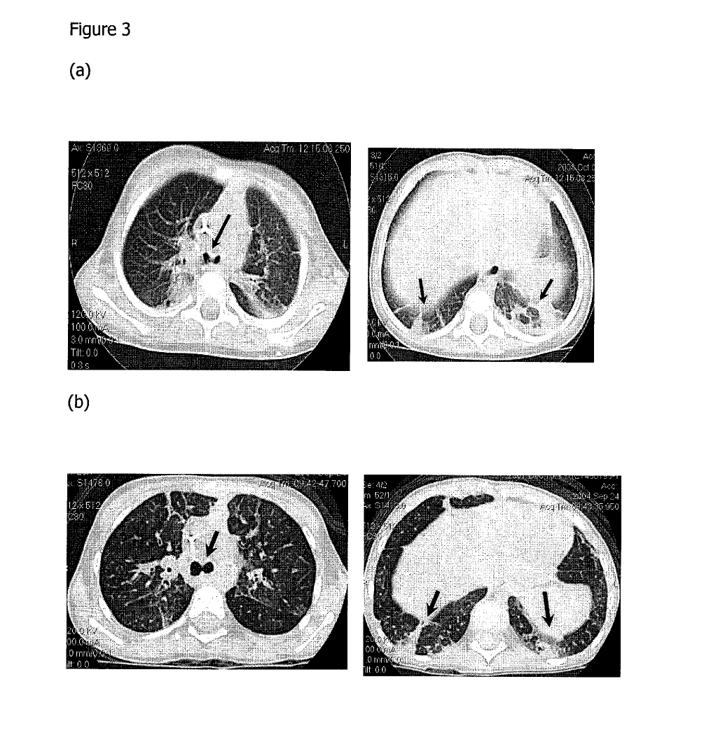

Initial management: Suspension laryngoscopy with CO₂ laser for debulking papillomas was performed ×6 (locally) and ×2 (larger tertiary centre) (FIG. 1b). Her lesions were described as “confluent papillomas covering the larynx, trachea, proximal main bronchi & proximal esophagus”. CT scan of the chest suggested distal spread of the papillomas (FIG. 3a). Palliation was discussed with the mother.

Adjuvant therapy: Medical management was initiated 6 months into treatment. Cidofovir was started (4.1 mg/kg/dose) every 4 weeks without effect. The regime was stepped-up to q2 weeks and Interferon α2b was added (5 MU/m² 3× weekly). Although her disease had ‘stabilized’ for 3 months (no surgical interventions required) her cidofovir dose was stepped down to q3 weeks due to concerns about renal toxicity and the lesions worsened. The cidofovir dose was stepped-up again (q2 weeks) but the disease continued to progress (FIG. 1c).

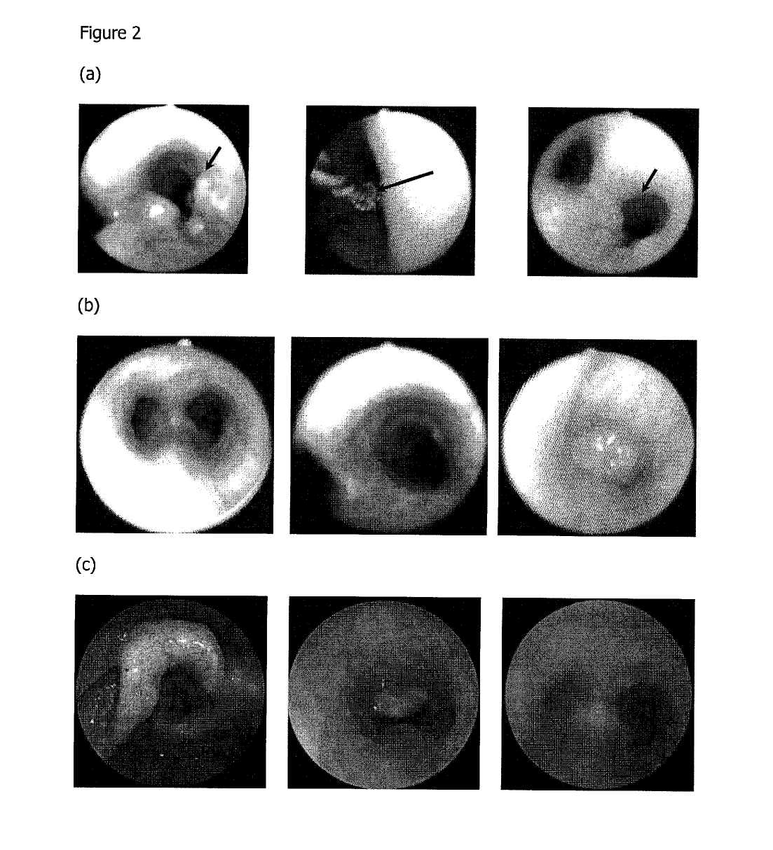

Topical and nebulized cidofovir: The patient was started on topically applied cidofovir via flexible bronchoscopy q2 weeks in addition to continuing the intravenous (systemic) dose. A cytology brush was soaked in cidofovir 10 mg/ml solution and advanced via the bronchoscope to the lower airway papillomas (FIG. 2a). We elected to start nebulized cidofovir to reduce the frequency of Bronchoscopic procedures and to intensify treatment. Cidofovir 10 mg/ml 4 ml (total dose 40 mg) was nebulized via Pari-nebulizer® 3× weekly but complications ensued (bleeding and protracted airways inflammation). The dose was halved (5 mg/ml) with resolution of the complications and reduction of the burden of the papillomas (FIG. 2b). After 6 months of nebulized cidofovir, the dose was reduced to 2× weekly.

Current status: A large papilloma was surgically debulked (microdebridment) and topical cidofovir was topically applied to the base of the lesion. Her disease has remitted and she has been successfully decannulated. She undergoes surveillance bronchoscopy q3 months and continues on nebulized cidofovir 5 mg/ml, 4 cc 2× weekly (FIGS. 2c and 3b).

While the preferred embodiments of the invention have been described above, it will be recognized and understood that various modifications may be made therein, and the appended claims are intended to cover all such modifications which may fall within the spirit and scope of the invention.

1. A method of treating recurrent respiratory papillomatosis comprising orally administering a solution of 2.5-20% cidofovir as droplets having a mean size of about 1 micron to about 20 microns to an individual in need of such treatment.
